(19) United States
(12) Patent Application Publication
(10) Pub. No.: US 2021/0380636 A1
(43) Pub. Date: Dec. 9, 2021

(54) PROCESS FOR PURIFYING C1-INH

(71) Applicant: CSL BEHRING GMBH, Marburg (DE)

(72) Inventors: Anna Kornilova, Goettingen (DE); Heike Nicole Wilka, Lahntal (DE)

(73) Assignee: CSL BEHRING GMBH, Marburg (DE)

(21) Appl. No.: 17/286,098

(22) PCT Filed: Oct. 17, 2019
(86) PCT No.: PCT/EP2019/078139

§ 371 Date: Apr. 16, 2021

(30) Foreign Application Priority Data
Oct. 17, 2018 (EP) 18201032.2

(51) Int. Cl. C07K 1/20; C07K 14/81; B01D 15/32; B01D 15/42

(52) U.S. Cl. CPC C07K 1/20; B01D 15/426; B01D 15/327; C07K 14/8121

(57) ABSTRACT

The present invention relates to a process for purifying C1-esterase inhibitor (C1-INH), and more in particular a C1-INH concentrate.

The present invention relates to a process for purifying C1-esterase inhibitor (C1-INH), and more in particular a C1-INH concentrate.

C1-INH, a protein of the pathway of complement activation, is an inhibitor of proteases present in the plasma, which controls C1-activation by forming covalent complexes with activated C1r and C1s. It also “controls” important blood coagulation enzymes, such as plasma prekallikrein, factors XI and XII, but also plasmin.

C1-INH deficiency is for instance associated with hereditary angioedema (HAE) caused by lack of C1-INH (HAE type I) or a reduced activity of C1-INH (HAE type II). C1-INH deficiency may also be caused by consumption of C1-INH due to neutralisation of enzymes generated when blood comes into contact with surfaces such as in a heart-lung machine, but also in disease courses initiating the coagulation cascade, such as immune complexes appearing in the context of chronic, in particular rheumatic disorders. Currently, C1-INH protein replacement must be considered as the gold standard in the prevention or treatment of acute HAE. This holds particularly true for commercially available human blood plasma derived C1-INH, which reportedly has a more natural functionality than a commercially available recombinant C1-INH produced in transgenic rabbits which is not identical to the human C1-INH protein (Feussner et al., Transfusion 2014 October; 54(10):2566-73). Further therapeutic applications that have been considered include the use of C1-INH in prevention, reduction and/or treatment of ischemia reperfusion injury (cf. WO 2007/073186).

Isolation and/or purification of C1-INH from human blood plasma is a known but more or less expensive and in particular most often a very time consuming process. Many prior art processes, such as e. g. described in Haupt et al., Eur. J. Biochem. 1970; 17:254-261; Reboul et al., FEBS Letters 1977; 79(1):45-50 are too complicated, associated with insufficient yields, and/or take too long to be amenable to a technical scale. Other prior art processes, such as e.g. described by Vogelaar et al. Vox Sang 1974; 26:118-127 have other drawbacks.

The different methods proposed for producing C1-INH from blood plasma include various separation methods such as affinity chromatography, cation exchange chromatography, anion exchange chromatography, gel filtration, precipitation, and hydrophobic interaction chromatography. Using any of these methods alone is generally insufficient to purify C1-INH, and in particular C1-INH concentrates, sufficiently, hence various combinations thereof have been proposed in the prior art.

EP 0 698 616 B describes the use of anion exchange chromatography followed by cation exchange chromatography. EP 0 101 935 B describes a combination of precipitation steps and hydrophobic interaction chromatography in a negative mode to arrive at a 90% pure C1-INH preparation at a yield of about 20%. U.S. Pat. No. 5,030,578 describes PEG precipitation and chromatography over jacalin-agarose and hydrophobic interaction chromatography in a negative mode. WO 01/46219 describes a process involving a first and a second anion exchange.

Today, there are four commercially available C1-INH concentrates for treatment of angioedema, three of which are plasma derived. One of these plasma derived C1-INH concentrates is sold under the trademark Berinert®. These C1-INH concentrates are prepared according to different proprietary processes, wherein the process to manufacture Berinert® involves a step of hydrophobic interaction chromatography (HIC) but in a negative mode (cf. in Feussner et al., Transfusion 2014 October; 54(10):2566-73).

In more general terms, HIC separates molecules based on their hydrophobicity and is used for purifying proteins while maintaining biological activity. Molecules, and more in particular proteins disposing of hydrophobic and hydrophilic regions are applied to an HIC column in a high-salt buffer. The salt in the buffer reduces the solvation of sample solutes. As solvation decreases, hydrophobic regions that become exposed are adsorbed by the media, or retained by and/or bound to the stationary phase. The more hydrophobic the molecule, the less salt is needed to promote binding. Usually a decreasing salt gradient is then used to elute samples from the column in order of increasing hydrophobicity. This mode of using HIC with respect to a molecule by first binding the molecule to a stationary phase and then eluting it will in the following be referred to as “positive mode”.

In the specific case of C1-INH, HIC has however not been used in this way, i.e. not in a “positive” or “binding” mode. This is because in the case of C1-INH HIC has been described to take advantage of the marked hydrophilicity of the C1-INH. Whereas other proteins are retained on the (hydrophobic) column, C1-INH remains in the mobile phase. This prior art technique of using HIC to purify C1-INH will in the following be referred to as “negative” or “flow through” mode. HIC in the flow through mode is how the prior art uses HIC for purifying C1-INH. The inventors are not aware of any description of using HIC in a different manner for purifying C1-INH. For instance, flow through was described as the core of the invention of EP 0 101 935. U.S. Pat. No. 5,030,578 also describes HIC under such conditions that C1-INH is not retained by the column (flow through mode), referring to Nilsson and Wiman, Biochimica et Biophysica Acta 1982; 705(2):271-276 in this context, which also describes HIC in a flow through mode. And more recently also Kumar et al., J. Bioproces Biotech 2014; 4(6) (DOI: 10.4172/2155-9821.1000174) describes an intermediate purification step of C1-INH involving HIC in a flow through or negative mode: The authors considered a 0.8 M ammonium sulphate concentration to be optimal to get purified C1-INH in the flow through fraction and to separate it from other plasma proteins. The C1-INH concentrate so obtained required further purification.

According to the aforementioned prior art, the starting material for HIC to purify C1-INH can be obtained in different ways, involving steps such as cryoprecipitation, ion exchange chromatography, fractioned precipitation and/or combinations thereof, wherein fractioned precipitation is known to be used on a technical or industrial scale, namely in the manufacture of Berinert® (wherein HIC is preceded by ammonium sulphate precipitations cf. Feussner et al., Transfusion 2014 October; 54(10):2566-73). According to EP 0 101 935 fractioned precipitation using liquid ammonium sulphate as a precipitant is carried out until the solution comprises 60% ammonium sulphate. Thereafter, the precipitated C1-INH is taken up with an aqueous solution containing the precipitant, in this case ammonium sulphate, at a concentration at which the C1-INH does not precipitate. Although this process has been brought to a technical scale, it still requires important resources, e. g. in form of time, space and material.

Human blood plasma is generally hard to come by in sufficient amounts to satisfy existing needs. It is therefore of utmost importance to come by with more efficient and in particular less time-consuming processes helping safeguarding optimal use thereof. The present invention accordingly aims at providing a more efficient and less time-consuming process for purifying C1-INH using hydrophobic interaction chromatography.

The aforementioned problem is solved by a process for purifying C1-INH using hydrophobic interaction chromatography (HIC), which comprises the steps of:
(i) loading a solution containing C1-INH dissolved therein onto a hydrophobic interaction chromatography column comprising a stationary phase under first conditions under which C1-INH binds to the stationary phase,
(ii) applying second conditions so as to elute C1-INH by means of a mobile phase.

Quite surprisingly in view of the prior art, the inventors have found that binding C1-INH to the stationary phase in an HIC enables a comparably huge economy of time and material. First, an HIC column used in the positive or binding mode may be loaded with a substantially higher amount of C1-INH containing starting material (inventors found up to about 4 times more) than an HIC column of essentially the same volume used in the flow through or negative mode to purify C1-INH. Hence less stationary phase material is necessary, leading to economy of column material plus space, and of course less volume of the aqueous solution containing C1-INH to be run through the column. Alternatively, larger volumes of C1-INH-containing starting material can be loaded on an existing-size column, resulting in a time-saving process. Second, binding C1-INH enables washing of the bound C1-INH, prior to eluting the C1-INH from the column. Third, HIC in a binding mode or positive mode enables using high flow rates and hence the purification of C1-INH in a much quicker time as compared to HIC in a flow through or negative mode, wherein the C1-INH interacts with, but does not bind to the stationary phase of the HIC column, i.e. wherein time is needed for a separation along a comparably long column at a slow flow rate.

In addition thereto, the inventors have found that, when working with a solution obtained by fractional precipitation using ammonium sulphate, initial material concentration by means of fractional precipitation including a precipitation of C1-INH using 60% ammonium sulphate and taking up C1-INH in an aqueous solution comprising the precipitant ammonium sulphate preceding purification using HIC in a binding or positive mode according to the invention becomes unnecessary. This initial material concentration step is required for a prior-art HIC usage in a negative mode for an efficient C1-INH purification. According to the present invention, however, the filtrate comprising just 40% ammonium sulphate of an earlier precipitation step may be used directly without loss of quality, which again leads to a more efficient manufacturing process by saving even more time, material and space in an otherwise established and well-understood process.

The present invention uses “a solution containing C1-INH dissolved therein”, and not a solution from which C1-INH precipitates. This means in other words that the first conditions must be chosen so as to avoid the occurrence of protein precipitation.

In the context of the present invention, “binds” to the stationary phase is to be understood as meaning is adsorbed by or retained on the stationary phase without the structural integrity of C1-INH being affected, preferably not by covalent bonds or chemisorption, but rather by physisorption.

The stationary phase is a matrix material, such as e.g. an agarose, a cross-linked agarose (sold under various trade names, such as Sepharose®), a hydrophilic polymer, e.g. polymethacrylate, which is respectively substituted with hydrophobic ligands such as
linear alkyl, e.g. ethyl, butyl, octyl,
ramified alkyl, e.g. t-butyl,
aryl, e.g. phenyl, or
cycloalkyl, e.g. hexyl.

Preferred matrix materials are those substituted with butyl or phenyl, more preferably cross-linked agarose substituted with butyl or phenyl, most preferably with phenyl. The matrix material may be presented in various forms, such as beads, or in the form of sticks, membranes, pellets, and so on. Cross-linked agarose in beaded form for use in various types of chromatography including HIC is also known under the tradename Sepharose®, of which various grades and chemistries are available. Particularly preferred types of matrix material are Phenyl Sepharoses®. Examples of commercially available matrix materials are hydrophobic interaction chromatography media sold under the names Capto™ Octyl, Capto™ Butyl, Capto™ Phenyl (high sub), Octyl Sepharose® 4 Fast Flow, Butyl Sepharose® 4 Fast Flow, Butyl-S Sepharose® 6 Fast Flow, Phenyl Sepharose® 6 Fast Flow® (low sub), Phenyl Sepharose® 6 Fast Flow® (high sub), Butyl Sepharose® High Performance, HiScreen™ Capto™ Butyl HP, Phenyl Sepharose High Performance®, all sold by GE Healthcare; Macro-Prep Methyl®, Macro-Prep t-Butyl®, both sold by BIO-RAD; or Toyopearl® Ether-650S, Toyopearl® Ether-650M, Toyopearl® PPG-600M®, Toyopearl® Phenyl-650S, Toyopearl® Phenyl-650M, Toyopearl® Phenyl-650C, Toyopearl® Phenyl-600M, Toyopearl® Butyl-650S, Toyopearl® Butyl-650M, Toyopearl® Butyl-650C, Toyopearl® Butyl-600M, Toyopearl® SuperButyl-550C, Toyopearl® Hexyl-650C, TSKgel® Ether-5PW (20), TSKgel® Ether-5PW (30), TSKgel® Phenyl-5PW (20), TSKgel® Phenyl-5PW (30), all sold by Tosoh. Among the aforementioned commercially available matrix materials, Phenyl Sepharose® 6 Fast Flow (low sub) and HiScreen™ Capto™ Butyl HP are particularly preferred, wherein the former is more preferred than the latter.

The first conditions are conditions which facilitate binding of the hydrophobic portion of C1-INH to the stationary phase, preferably in the presence of or by addition of one or more specific salts to the C1-INH containing solution.

The second conditions are conditions which allow for the elution of C1-INH from the stationary phase and consequently collection of purified C1-INH in an eluate. Several types of elution exist, e. g. elution with an elution buffer comprising a stepwise decreasing salt concentration, a continuously decreasing salt concentration, elution using a pH gradient, elution using a temperature gradient, or combinations thereof. Still further types of elution exist, wherein solvents less polar than water are used as elution buffers, e. g. aqueous solutions comprising ethanol, PEG, 2-Propanol, or the like. Also a gradient of a calcium chelating compound (such as EDTA, citrate, malonate, etc.) may be used as an elution buffer.

Preferably the first conditions are that the mobile phase comprises an anti-chaotropic salt, preferably sodium sulphate or ammonium sulphate, most preferably ammonium sulphate in a first concentration at which C1-INH binds to the stationary phase and the second conditions are that the mobile phase comprises the anti-chaotropic salt, preferably sodium sulphate or ammonium sulphate, most preferably ammonium sulphate in a second concentration at which C1-INH elutes. Sodium sulphate and in particular ammonium sulphate are commonly used, reliable and in particular well-established anti-chaotropic salts in HIC and are hence preferred.

The concentration of ammonium sulphate that may be added depends on the protein concentration of the sample. The higher the protein concentration, the lower the possible ammonium sulphate concentration of the sample, i.e. the lower the ammonium sulphate concentration at which protein precipitation starts to occur. Dilution of the sample makes it possible to add a higher amount of ammonium sulphate. An optimum protein concentration when using ammonium sulphate as an anti-chaotropic salt is in the range of 0.1 to 3 mg/mL protein. Other concentrations ranges may apply when anti-chaotropic salts other than ammonium sulphate are used.

The transition from the first concentration to the second concentration may be achieved by means of a concentration gradient or by means of a step elution, wherein step elution is preferred, as step elution has the advantage to save time and is easier to implement in a large scale manufacturing process. Step elution as used herein is intended to mean a sudden transition from the first to the second concentration instead of a continuous transition as in a concentration gradient, wherein the concentration is gradually lowered.

The specific first and second concentrations depend on the circumstances, i. e. types of stationary phase used, pH, salt, etc. Without wanting to be limited by the following numbers, which merely serve as an example, the first concentration may for instance be situated somewhere between 1 to 2 M, and the second concentration below the first concentration e.g. between 0.0 and 1.4 M.

When a phenyl substituted Sepharose® gel such as Phenyl Sepharose® 6 Fast Flow (low sub) by GE Healthcare is used as stationary phase and ammonium sulphate is used as chaotropic salt, the first concentration is preferably above a concentration X in a range of about 1.1 M to about 1.4 M (e. g. above a concentration X in the range of about 155 to about 180 mg/ml ammonium sulphate), preferably in a range of about 1.2 M to about 1.3 M (e. g. above a concentration X in the range of about 160 to about 174 mg/ml), and the second concentration is below concentration X.

When butyl substituted Sepharose® gel such as HiScreen™ Capto™ Butyl HP by GE Healthcare is used as stationary phase, and ammonium sulphate is used as chaotropic salt, the first concentration is preferably above a concentration X in a range of about 0.9 M to about 1.0 M (e. g. a concentration X in the range of about 124 to about 131 mg/ml), and the second concentration of preferably ammonium sulphate is below concentration X.

When Phenyl-HP® or Capto Phenyl ImpRes® sold by GE Healthcare or Phenyl-650M® or Phenyl-600M® sold by Tosoh is used as stationary phase, and ammonium sulphate is used as chaotropic salt, the first concentration is preferably above a concentration X in a range of about 0.9 M to about 1.0 M (e. g. a concentration X in the range of about 124 to about 131 mg/ml), and the second concentration of preferably ammonium sulphate is below concentration X.

When different ammonium sulphate concentrations are used as first and second conditions, preferably the first concentration is about 181 mg/ml (1.37 M), and/or the second concentration is low enough to elute C1-INH from the stationary phase.

While the invention can be carried out with different starting materials containing C1-INH, it is preferred that the C1-INH concentrate used as a starting material is obtained by a process involving a fractional precipitation with a precipitant.

When the C1-INH concentrate used as a starting material is obtained by a process involving a fractional precipitation with a precipitant, the fractional precipitation may either (i) involve precipitation of C1-INH and taking up the precipitated C1-INH in a solution containing the precipitant at a concentration lower than necessary for a precipitation of C1-INH, or (ii) not involve precipitation of C1-INH, by providing a starting material wherein C1-INH is contained in a supernatant containing the precipitant used in a fractional precipitation at a concentration lower than necessary for a precipitation of C1-INH, wherein alternative (ii) is preferred.

The process according to the invention is preferably carried out at a pH in the range of 6 to 9, preferably 6.8 to 8.5, more preferably 7 to 7.5, and even more preferably at a pH of about 7.2.

While the inventive process according to the invention may in principle also be used to purify C1-INH produced in a different way, it is preferred that the process be carried out with recombinant C1-INH, transgenic C1-INH, or C1-INH derived from blood plasma, preferably human blood plasma.

The process according to the present invention may either be carried out in a column or in a batch format.

In the following, the present invention will be described in more details by means of figures and examples, wherein the figures depict the following:

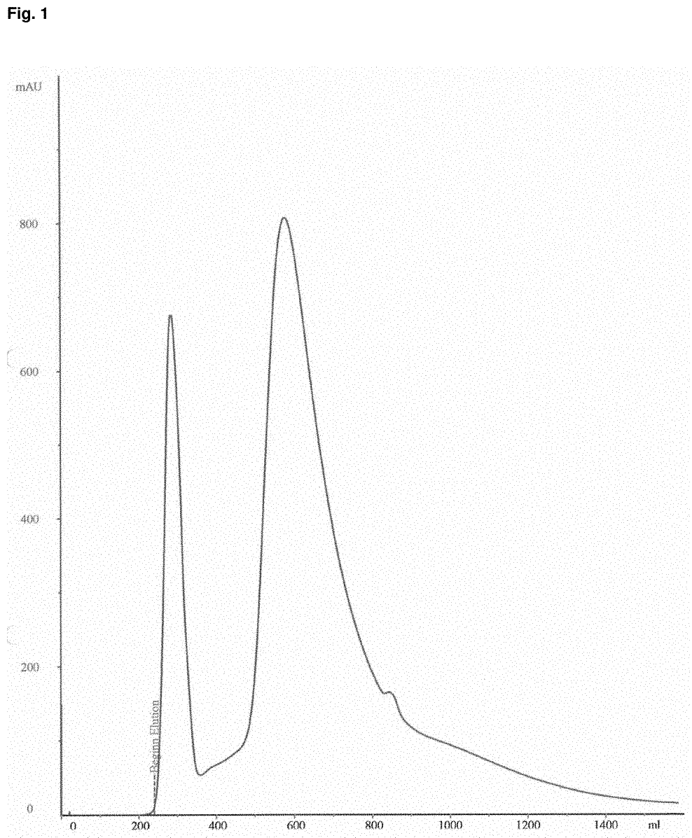

FIG. 1: chromatogram of a HIC carried out in a flow through or negative mode at normal load (“single load”);

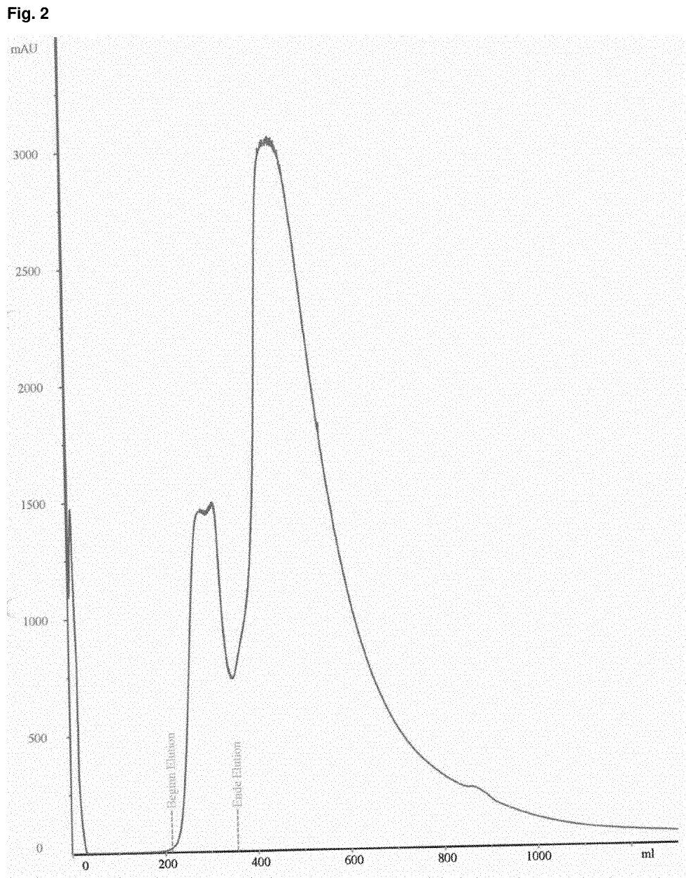

FIG. 2: chromatogram of a HIC carried out in a flow through or negative mode at a higher load than used in the prior art (“double load”);

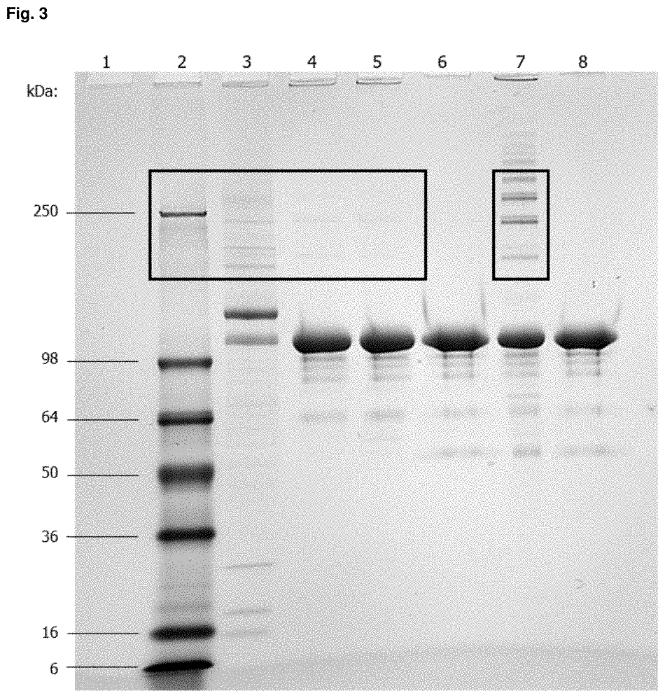

FIG. 3: an electrophoresis gel of eluate fraction samples of various HIC experiments including an experiment according to the prior art, a comparative example and experiments according to the present invention;

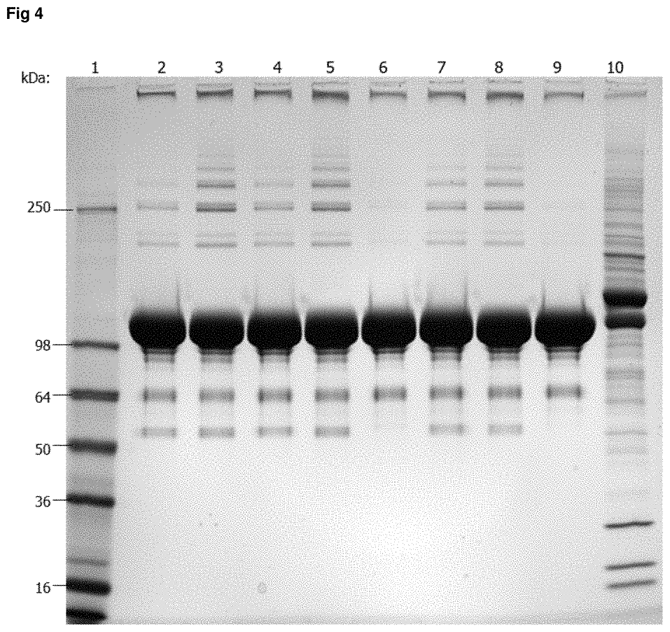

FIG. 4: an electrophoresis gel of eluate fraction samples of various HIC experiments to compare single and double loads in HIC according to the prior art;

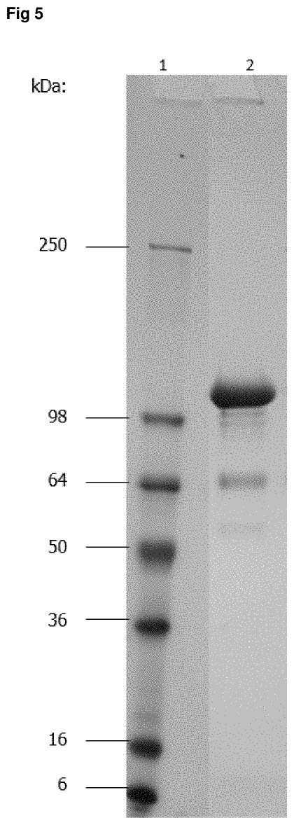

FIG. 5: an electrophoresis gel of an eluate fraction sample of another HIC experiment according to the present invention;

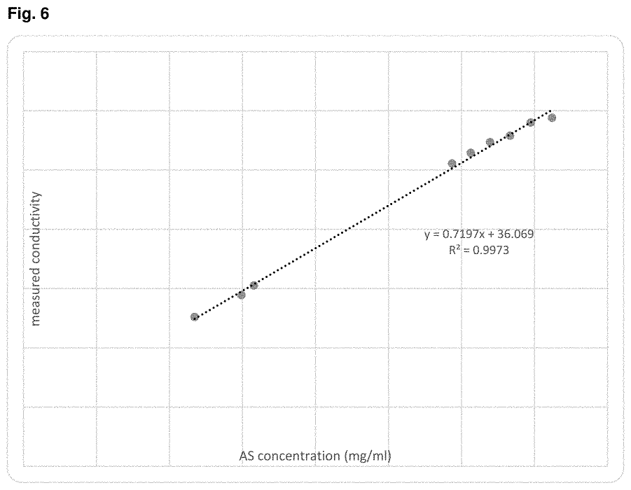

FIG. 6: a standard curve correlating sample conductivity with precipitant concentration;

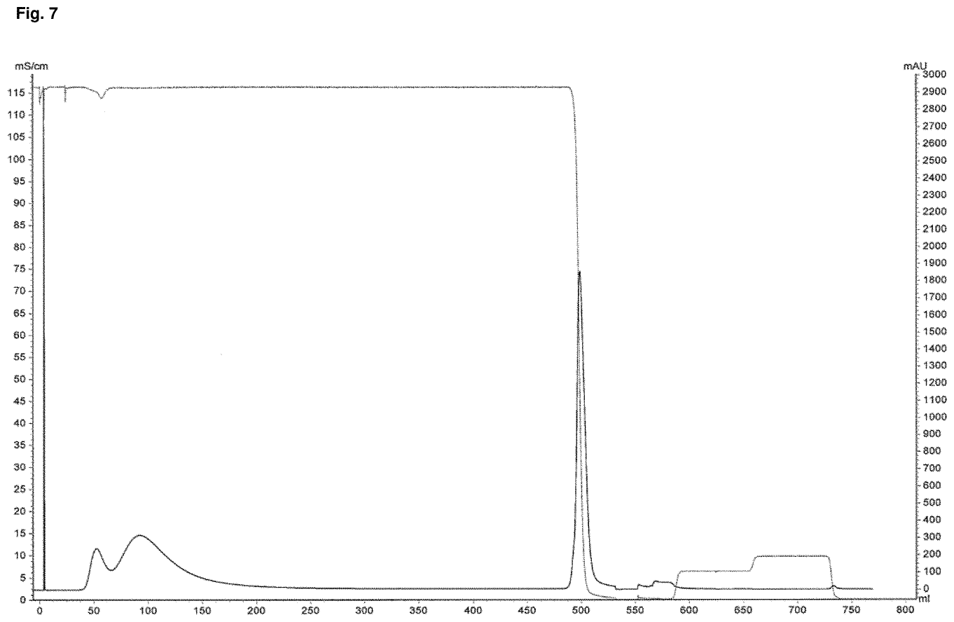

FIG. 7-11: various chromatograms of HIC carried out in accordance with the prior art and according to the invention.

In the context of the present invention, the following definitions apply:

In the claims and in the description of the invention “C1-INH” and “C1-INH concentrate” are concurrently used to designate concentrates containing the protein C1-esterase inhibitor and liquid concentrates containing the protein C1-esterase inhibitor. When referring to the technical background and/or prior art, “C1-INH” may also mean the protein as such, e.g. in the context of discussing C1-INH deficiency.

Throughout the present application/patent
“HIC” stands for hydrophobic interaction chromatography;
“negative mode” or “flow through mode”, or “flow through” HIC designates a way of carrying out HIC under conditions under which C1-INH does not bind to the stationary phase of the HIC column;
“binding mode”, “binding and elution” or “positive mode” stands for a HIC first carried out under conditions under which C1-INH binds to the stationary phase of a HIC column and then under conditions under which C1-INH is eluted from the HIC column;
“binds to the stationary phase” is intended to mean is adsorbed by or retained on the stationary phase without the structural integrity of C1-INH being affected, preferably not by covalent bonds or chemisorption, but rather by physisorption;
“WFI” means “water for injection”;
“single load” designates a usual load, and in the present context more in particular an essentially maximal load at which a satisfactory purification of C1-INH by means of HIC when carried out in a flow through mode occurs, wherein such a usual “single load” may vary depending on the circumstances, e. g. starting material used, the chromatographic matrix used, etc., and wherein such a usual “single load” has a numerical value of about 6 to 9, preferably about 7 to 8 and most preferably of about 7.5 mg protein/ml chromatography gel, when using a phenyl substituted Sepharose® as chromatographic matrix and when using a C1-INH concentrate as a starting material which was generated by fractional precipitation and re-dissolution of C1-INH as described in prior art EP 0 101 935;
“double load” designates the doubled or 2-fold amount of a single load, and in the present context more in particular a load at which purification of C1-Inh by means of HIC when carried out in a flow through mode is not satisfactory anymore;
“concentration gradient” designates the gradual variation of the concentration of a dissolved substance in a solution from a higher concentration to a lower concentration,
“step elution” means a sudden transition from the first to the second concentration instead of a continuous transition as in a concentration gradient, wherein the concentration is gradually lowered;
“%” means “% by weight” unless otherwise stated;
“precipitant” is an agent triggering precipitation of proteins; the precipitant may also serve as an anti-chaotropic agent or salt;
“anti-chaotropic agent” or “anti-chaotropic salt” as used herein is intended to refer to one or more salts capable of making C1-INH so hydrophobic in aqueous solution that it will bind to the stationary phase;
“eluate fraction” designates a fraction of the mobile phase stream emerging from the chromatographic column irrespective of whether specific analytes comprised therein were previously bound to or retained by the stationary phase (as in a positive mode as described herein) or not (as in a negative mode as described herein).

In the following, the present invention will be explained in more detail by making reference to the figures.

FIGS. 1 and 2 are respectively a chromatogram of a negative mode HIC using a C1-INH concentrate obtained by fractional precipitation according to the prior art, i.e. using C1-INH precipitated and then re-dissolved as a starting material. FIG. 1 shows the chromatogram of a “single load” as used in the prior art, and FIG. 2 that of a “double load” for comparison. The first peak (respectively starting at 200 ml eluate) in the chromatograms respectively represents the flow through fraction containing C1-INH. From FIG. 1 it can be seen that the first peak is a rather sharp single peak essentially not overlapping with other peaks, whereas from FIG. 2 it can be seen that the first peak in fact consists of several overlapping peaks. Also, the first overlapping peaks at their end overlap with the following, much larger peak to a higher extent than the single sharp peak in the single load experiment depicted in FIG. 1. This indicates that the “single load” used to purify C1-INH using HIC in a flow through or negative mode cannot be doubled without drawbacks regarding purity. FIGS. 1 and 2 thus illustrate what is to be understood by a “single load” and a “double load” in the context of the present invention: A single load is the load of C1-INH containing starting material, which results in essentially a single peak attributable to C1-INH which is essentially not overlapping with other peaks in the chromatogram and thus enables obtaining an essentially pure C1-INH eluate in an HIC carried out in accordance with the prior art, i. e. in a flow through or negative mode, wherein the double load of the same starting material under otherwise essentially the same conditions does not result in essentially a single peak attributable to C1-INH not essentially overlapping with other peaks in the chromatogram, i.e. wherein the double load does not enable a scale up without essential quality losses as regards the purity of the desired C1-INH eluate in comparison to the single load.

FIG. 3 is an SDS-PAGE gel (Tris-Glycine gel, 1.5 mm thick, gradient 8-16%, max. voltage 150 V, run time: 90 min.) of samples of various C1-INH containing HIC eluate fractions from HIC experiments, all using a C1-INH concentrate as a starting material which was generated by fractional precipitation and re-dissolution of C1-INH as described in prior art EP 0 101 935. To allow for better comparison, samples loaded onto the gel comprise approximately same amounts of protein.

In the gel represented in FIG. 3, lane 3 is C1-INH concentrate used as a starting material. It can be seen that the starting material contains other proteins of higher and lower molecular weight. Lane 4 is the C1-INH containing eluate fraction of HIC from the Berinert® manufacturing process, i.e. from an industrial scale process according to the prior art. The band with the highest intensity in lane 4 is C1-INH, weighing approximately 105 kD. As can clearly be seen, high molecular weight components cannot be detected in this fraction.

Lanes 5 and 7 are C1-INH containing eluate fractions of HIC experiments in a flow through. The sample of lane 5 is taken from a single load experiment, and that of lane 7 from double load experiment. High molecular weight impurities are detectable in the starting material (lane 3), in the Berinert® production sample (lane 4) and in the respective single load and double load flow through samples (lanes 5, 7). Bands attributed to high molecular weight impurities in lanes 3, 4, 5, 7 are highlighted by boxes in FIG. 3. Bands attributed to high molecular weight impurities are comparably weak in lanes 4 and 5, more pronounced in lanes 3 and 7. As can clearly be seen from lane 7, the double load eluate fraction contains more high molecular weight impurities than detectable in the single load eluate fraction (cf. lane 5) and in the eluate fraction from the Berinert® manufacturing process (cf. lane 4). This finding was verified by carrying out still further experiments with starting materials from different plasma preparations, the results of which are shown in FIG. 4 discussed further below. This clearly shows that carrying out HIC in the flow through or negative mode according to the prior art is limited with regard to the maximal load of a column enabling a purification of a C1-INH concentrate without quality losses. The single load used in these experiments corresponds to a load of 7.5 mg protein/ml chromatography gel.

Lanes 6 and 8 in FIG. 3 are C1-INH containing eluate fractions of HIC experiments according to the present invention, i. e. wherein HIC was carried out in a binding and elution, or positive mode. The eluate fraction of lane 6 in FIG. 3 is from a single load experiment, and the eluate fraction of lane 8 in FIG. 3 from a double load experiment (using 15 mg protein/ml chromatography gel). The gel shows that impurities having a weight above that of C1-INH, i.e. above 105 kD, could not be detected in the respective eluate fraction also when a double load had been applied to the column (cf. lane 8 in FIG. 3).

Thus lane 6 in FIG. 3 demonstrates that HIC according to the present invention provides a viable alternative solution to get rid of high molecular weight impurities in C1-INH concentrates, yielding a product with less high molecular weight impurities than the prior art. Irrespective thereof, lane 8 demonstrates that HIC according to the present invention is less limited with regard to the maximal load of a column enabling to arrive at a purification of a C1-INH concentrate essentially without quality losses than the prior art. In other words: Inventors could show that the maximal load of a column enabling to arrive at a purification of a C1-INH concentrate can at least be doubled by using the positive or binding mode according to the present invention without the drawbacks as regards purification as otherwise inevitable when using HIC in the negative or flow-through mode in accordance with the prior art.

FIG. 4 is an SDS-PAGE gel (Tris-Glycine gel, 1.5 mm thick, gradient 8-16%, max. voltage 150 V, run time: 90 min.) with samples of various C1-INH containing HIC eluate fractions from HIC experiments according to the prior art, i. e. in a flow through or negative mode, using a C1-INH concentrate as a starting material which was generated by fractional precipitation and re-dissolution of C1-INH as described in prior art EP 0 101 935. To allow for better comparison, samples loaded onto the gel comprise approximately same amounts of protein. In the gel of FIG. 4, lane 1 is marker, lanes 6 and 9 respectively are Berinert® final product samples from different charges, and lane 10 a sample of a typical starting material. Lanes 2, 4 and 7 represent eluate fractions of HIC carried out with a single load, and lanes 3, 5 and 8 represent eluate fractions of HIC carried out with a double load, i.e. twice the amount of C1-INH containing starting material. High molecular weight impurities are detectable in every sample, including the final product samples (cf. lanes 6, 9 in FIG. 4), wherein the impurities are difficult to detect in the latter. Comparison of intensities of the bands of single and double load samples reveals that the double load samples contain more high molecular weight impurities of than the single load samples. The gel in FIG. 4 in other word provides further evidence regarding the limitation of the process according to the prior art as regards the maximal load allowing for a purification of C1-INH concentrates.

Inventors believe that the maximal load of a column enabling a purification of a C1-INH concentrate essentially without quality losses by using the present invention is lastly limited by the C1-INH containing starting material loading capacity of the chromatographic matrix, until the matrix starts loosing C1-INH. In the case of Phenyl Sepharose®, the loading capacity of the column when using C1-INH containing starting material consisting of supernatant or filtrate of a precipitate fraction containing 40% of ammonium sulphate was found to be about 4-fold or even 4.4-fold the single load of C1-INH containing starting material consisting of a re-dissolved 60% ammonium sulphate precipitate applied in flow through (according to the prior art) to be able to arrive at a purified C1-INH concentrate. Hence on a production scale, the load may in principle not only be doubled as compared to the prior art, but may even be more than twice the load currently used. This means that important economies regarding column volume and/or stationary phase material may indeed be realized thanks to the present invention, and this without any quality losses.

Inventors also found that the process according to the invention can be carried out at a much higher flow rate as compared to using HIC in a flow through or negative mode to arrive at the desired purified concentrate without any quality losses. The economy is rather important: While a conventional HIC run at the scale currently used in the Berinert® process usually takes 42.6 hours, an optimized run using the present invention can be carried out in as little as 6 hours when using a single load, cutting down the HIC process step and thus the overall process time by 36.6 hours. When using a double load, a run can be carried out in 6.6 hours, and the ability to use a double load may cut down the overall process time by as much as 78.6 hours.

FIG. 5 is an SDS-PAGE gel (Tris-Glycine gel, 1.5 mm thick, gradient 8-16%, max. voltage 150 V, max. amperage 35 mA, run time: 90 min.) of a C1-INH containing eluate fraction from a HIC experiment wherein the starting material was generated by fractional precipitation at precipitant concentrations lower than necessary to precipitate C1-INH, i. e. without precipitation of C1-INH as in the prior art, namely the supernatant or filtrate of a precipitate fraction containing 40% of ammonium sulphate. The most intensive band is again C1-INH, and also here higher molecular weight components could not be detected. This is remarkable because the supernatant or filtrate of the 40% ammonium sulphate precipitate comprises more impurities than the solution generated from a 60% ammonium sulphate precipitate as in the prior art. This also means that the process according to the present invention has the additional advantage to enable carrying out the prior art process without precipitating C1-INH in a fractional precipitation and re-dissolving it prior to carrying out an HIC purification.

Inventors thus also found that the claimed process enables cutting down process times even more by omitting the precipitation of C1-INH in a fractional precipitation and the re-dissolution of C1-INH preceding HIC. This enables to save an additional 9.2 hours otherwise needed therefore. The process according to the invention thus enables to save even more process time, namely 45.8 hours when running single loads, and even up to 97 hours when running the process with a double load.

As discussed above, the inventors believe that the maximal load of a column enabling a purification of a C1-INH concentrate essentially without quality losses by using the present invention is only limited by the C1-INH containing starting material binding capacity of the column, and that hence the load may not only be doubled as compared to the prior art, but may even be more than twice the load currently used. This means that even more important economies regarding column volume and/or stationary phase material and/or time than discussed above may in principle be realized thanks to the present invention, without quality losses, while possibly achieving an improvement in purity at the same time even on a production scale.

FIG. 6 shows a standard curve correlating sample conductivity with precipitant concentration. An anti-chaotropic salt is used as a precipitant, and mostly sodium or ammonium sulphate, wherein the latter is preferred. The concentration of the salt in a buffer solution can be correlated with its conductivity, as shown in FIG. 6 and discussed in more detail in the experimental section below. This enables proper analysis of corresponding samples for precipitant or rather anti-chaotropic salt concentrations.

FIGS. 7 to 11 are chromatograms obtained from HIC according to the prior art and according to the present invention, wherein respectively the axis of abscissa indicates the eluent volume exiting the column in ml, the left axis of ordinates indicates conductivity in mS/cm and the right axis of ordinates indicates absorbance in mAU. Conductivity can be directly linked to ammonium sulphate concentration of the eluent by means of the correlation coefficient determined as explained above.

FIG. 7 is a chromatogram resulting from a HIC according to the prior art. The starting material is a plasma derived C1-INH containing concentrate generated by fractional precipitation and dissolution of a precipitate as described in EP 0 101 935. The ammonium sulphate concentration remains constant at about 106 mg/ml for a while. This concentration is too low for retention of C1-INH by the stationary phase. The C1-INH containing peak is seen at about 50 ml eluent volume. A step elution of proteins other than C1-INH bound to the column at the initial ammonium sulphate concentration can be seen at around 500 ml eluent volume. It takes place when the ammonium sulphate concentration is suddenly decreased.

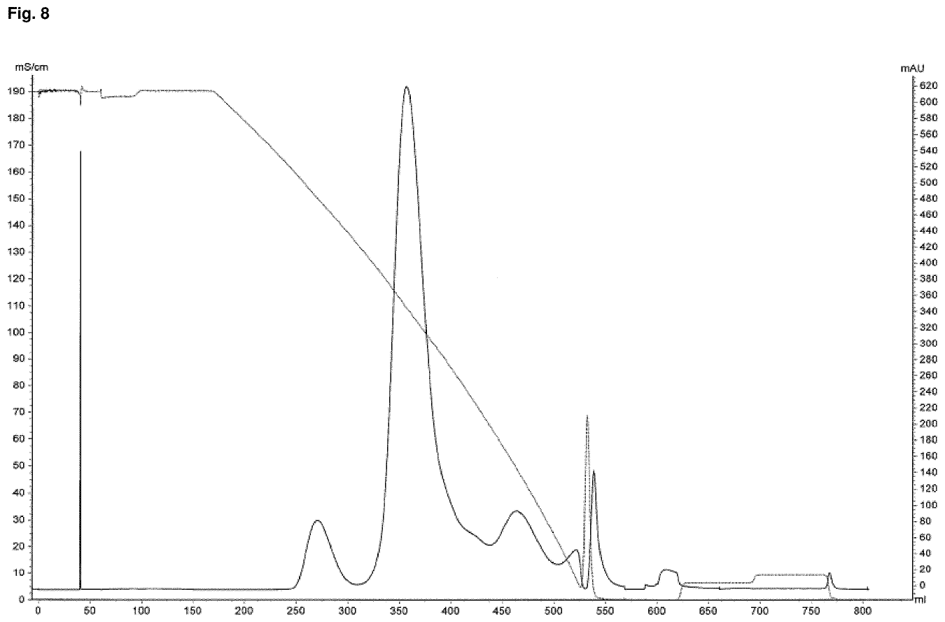

FIG. 8 is a chromatogram resulting from a HIC according to the present invention with elution by means of a concentration gradient. The starting material is a plasma derived C1-INH containing concentrate generated by fractional precipitation and dissolution of a precipitate as described in EP 0 101 935. The initial ammonium sulphate concentration is high enough for retention of C1-INH on the stationary phase until the ammonium sulphate concentration of the eluent is lowered to slightly below about 160 mg/ml. The corresponding peak attributed to C1-INH is seen at about 270 ml eluent volume.

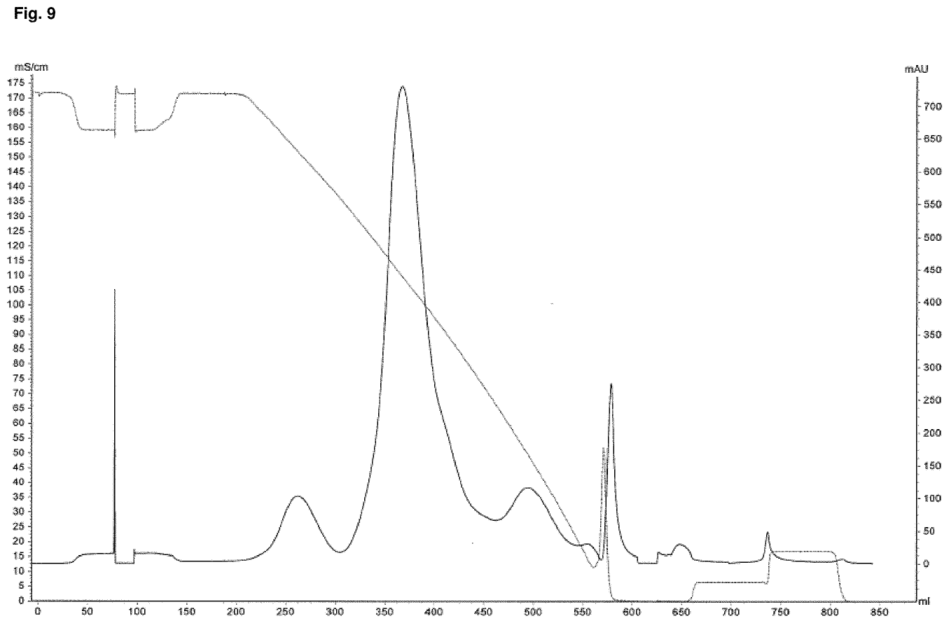

FIG. 9 is a chromatogram resulting from a HIC according to the present invention with elution by means of a concentration gradient. The starting material is a plasma derived C1-INH containing concentrate obtained from the supernatant or filtrate of a fractional precipitation with 40% ammonium sulphate. The initial ammonium sulphate concentration of the solution is high enough for retention of C1-INH on the stationary phase until the ammonium sulphate concentration of the eluent is lowered to slightly below about 160 mg/ml. The corresponding peak attributed to C1-INH is seen at about 270 ml eluent volume.

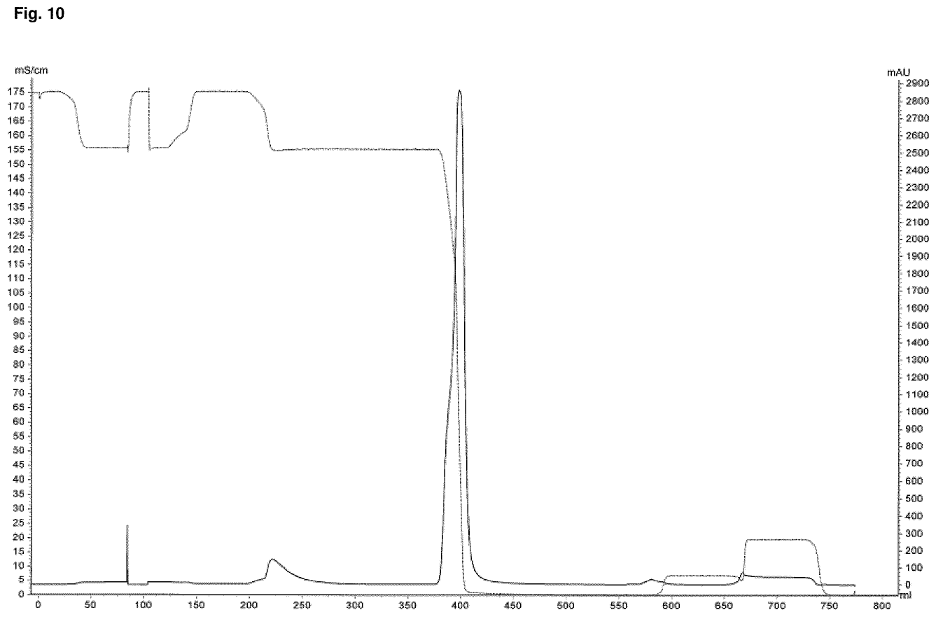

FIG. 10 is a chromatogram resulting from a HIC according to the present invention using a step elution instead of a concentration gradient. The starting material is a plasma derived C1-INH containing concentrate obtained from the filtrate of a fractional precipitation with 40% ammonium sulphate. The initial ammonium sulphate concentration of the solution is high enough for retention of C1-INH on the stationary phase until the ammonium sulphate concentration of the eluent is suddenly lowered.

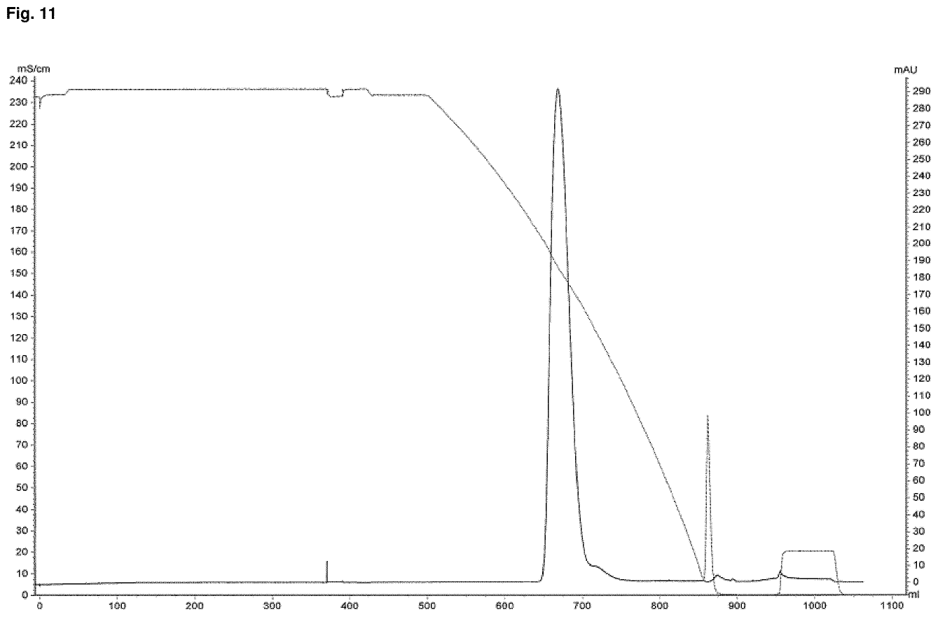

FIG. 11 is a chromatogram resulting from a HIC according to the present invention with elution by means of a concentration gradient. The starting material is Berinert® concentrate according to the prior art. The initial ammonium sulphate concentration of the solution is high enough for retention of C1-INH on the stationary phase until the ammonium sulphate concentration of the eluent is lowered to slightly below about 162 mg/ml. The corresponding peak attributed to C1-INH is seen at about 670 ml eluent volume.

While the inventors were concerned with improving the Berinert® manufacturing process described in the aforementioned prior art, it is evident that HIC in a positive mode also benefits other C1-INH purification processes. The invention is in other words clearly not restricted to being used in the process described in EP 0 101 935 or in the Berinert® manufacturing process, but also in other processes aiming to purify C1-INH concentrates using different starting materials previously involving a HIC step in the flow through mode or even in future processes yet to be designed to purify C1-INH concentrates of whatever origin (e.g. concentrates obtained from blood plasma, or C1-INH concentrates containing recombinant C1-INH obtained from transgenic animals, or C1-INH concentrates obtained by still different means).

### EXAMPLES

### Material and Methods

### I. Column A

Materials used:
a C1-INH sample derived from plasma respectively in the form of a semi-purified fraction;
Phenyl Sepharose® 6 Fast Flow (low sub) by GE Healthcare (a commercially available aromatic hydrophobic interaction chromatography (HIC) resin stored in 20% ethanol)
ammonium sulphate buffer:
   181 mg/mL (175-292 mg/mL) ammonium sulphate,
   25 mM Tris,
   pH 7.2±0.2
tris buffer:
   25 mM Tris
   pH 7.2±0.2
chromatography column, diameter: 1.6 cm (Äkta Avant, GE Healthcare)
UV spectrophotometer (unicorn);
conductivity meter.
1. Loading HIC column A: The Phenyl Sepharose® gel stored in 20% ethanol is washed thrice with water for injection (WFI). A 70% slurry of the washed Phenyl Sepharose® gel with WFI is prepared and placed in the chromatography column. Using WFI and a linear flow rate of 150 cm/h, the gel is packed to a gel bed height of about 18 cm (20±5 cm). The column is then tested by injecting 2.5% of the column volume 5% acetone (v/v). The column test is passed, provided the asymmetry is 0.8-1.8 and the theoretical number of plates is 2800.
2. Sample preparation: The plasmatic C1-INH sample to be purified is brought to an ammonium sulphate concentration of 181 mg/mL (175-292 mg/mL) and to a Tris content of 25 mM. The concentration of ammonium sulphate that may be added depends on the protein concentration of the sample. The higher the protein concentration, the lower the possible ammonium sulphate concentration of the sample, i.e. the lower the ammonium sulphate concentration at which protein precipitation starts to occur. Dilution of the sample makes it possible to add a higher amount of ammonium sulphate. An optimum protein concentration is in the range of 0.1 to 3 mg/mL protein. The sample comprises 25 mM Tris for pH adjustment. Following the addition of ammonium sulphate and Tris, the sample is adjusted to pH 7.2±0.2 by addition of 1 M NaOH or 1 M HCl and filtered over a 0.45 μm filter. Following measurement of the protein concentration, the loading of the column (in the case of column A) was calculated so as to reach a loading of at most 30 mg protein/mL gel. The protein concentration is determined by known methods based on measurements of the optical density (OD) of the respective sample at 280 nm.
3. Equilibration of the column: The column is equilibrated at a linear flow rate of 100 cm/h using≥3 column volumes ammonium sulphate buffer.
4. Loading the sample onto the column: The sample is loaded onto the column at a linear flow rate of 100 cm/h. The column is then washed with 3 column volumes ammonium sulphate buffer at the same flow rate.
5. Elution of C1-Inhibitor: The C1-INH is eluted at a linear flow rate of 100 cm/h over 20 column volumes by means of a gradient of ammonium sulphate buffer to Tris buffer. The complete elution is fractioned and then the non-reduced single fraction is loaded onto a Tris-glycine-gel and analyzed. Using the banding pattern it could be shown that the first peak is C1-INH.
6. Column regeneration: Regeneration of the column is carried out at a linear flow rate of 100 cm/h by subsequently using 3 column volumes WFI, 4 column volumes 0.1 M NaOH, 3 column volumes WFI.

### II. Column B

Materials used:
a C1-INH sample derived from plasma respectively in the form of a semi-purified fraction;
HiScreen™ Capto™ Butyl HP, GE Healthcare, Code 28-9782-42; diameter: 0.77 cm; gel bed height: 10 cm; gel volume: 4.7 ml
ammonium sulphate buffer:
   181 mg/mL (131-292 mg/mL) ammonium sulphate,
   25 mM Tris,
   pH 7.2±0.2
tris buffer:
   25 mM Tris
   pH 7.2±0.2
Äkta Avant, GE Healthcare, Unicorn, UV spectrophotometer, conductivity meter.
1. Sample preparation: The plasmatic C1-INH sample to be purified is brought to an ammonium sulphate concentration of 181 mg/mL (131-292 mg/mL) and to a Tris content of 25 mM. The concentration of ammonium sulphate that may be added depends on the protein concentration of the sample. The higher the protein concentration, the lower the possible ammonium sulphate concentration of the sample, i.e. the lower the ammonium sulphate concentration at which protein precipitation starts to occur. Dilution of the sample makes it possible to add a higher amount of ammonium sulphate. An optimum protein concentration is in the range of 0.1 to 3 mg/mL protein. The sample comprises 25 mM Tris for pH adjustment. Following the addition of ammonium sulphate and Tris, the sample is adjusted to pH 7.2±0.2 by addition of 1 M NaOH or 1 M HCl and filtered over a 0.45 μm filter. Following measurement of the protein concentration, the loading of the column (in the case of column B) was calculated so as to reach a loading of 7.5 mg protein/mL gel, i.e. column B was only tested with loads of 7.5 mg protein/ml chromatography gel. The protein concentration is determined by known methods based on measurements of the optical density (OD) of the respective sample at 280 nm.
2. Equilibration of the column B, loading the sample onto column B, elution of C1-INH and column regeneration are effected respectively in the same way as described above for column A.
   Calculation methods:
1. Determination of ammonium sulphate concentration for the elution of C1-INH: Conductivity, UV signals at 280 nm and 610 nm were recorded throughout a chromatography run. This enabled the inventors to assign a conductivity to the C1-INH peak in the chromatogram. A calibration line was created by preparing a buffer dilution series and measuring the corresponding conductivities. Measurements are shown in the following table 1, wherein AS stands for ammonium sulphate.

TABLE 1

| | |
|---|---|
| standard solution | measured |

| weight | solution | concentration | conductivity |
|---|---|---|---|
| AS (mg) | volume (ml) | g/l | mS/cm |
| 10 | 61 | 164 | 154.7 |
| 10 | 60 | 167 | 155.8 |
| 10 | 58 | 172 | 158.8 |
| 10 | 59 | 169 | 158.0 |
| 10 | 62 | 161 | 152.9 |
| 10 | 63 | 159 | 151.1 |
| 10 | 81 | 123 | 125.2 |
| 10 | 77 | 130 | 128.9 |
| 10 | 76 | 132 | 130.5 |

It could be shown by conversion of the conductivity into ammonium sulphate concentration that all C1-INH elutions took place at an AS concentration of between 160 mg/ml and 174 mg/ml when using the Phenyl Sepharose® matrix of column A and of between 124 and 131 mg/ml when using the HiScreen™ Capto™ Butyl HP matrix of column B. The corresponding calibration line allowing for determination of the AS concentration based on conductivity measurements is shown in FIG. 6.
2. Determination of the highest possible ammonium sulphate concentration without precipitation: A titration was carried out to determine the highest possible ammonium sulphate concentration, at which a retention or binding of C1-INH to the stationary phase is possible without protein precipitation. In this experiment a saturated ammonium sulphate was added to the C1-INH sample until a precipitation took place. The so determined highest possible ammonium sulphate concentration in the sample was 292 mg/mL. This was verified by conducting a run with the same concentration, in which it could be shown that C1-INH could be bound to and subsequently be eluted from the stationary phase (cf. Table 2 below, experiment 180619HW and FIG. 11).
3. Determination of the maximal protein loading capacity in comparison to the flow through process according to the prior art: To determine the maximal protein loading capacity in comparison to the flow through process according to the prior art, the Phenyl Sepharose® gel (column A) was loaded with starting material 1 under binding conditions until a UV signal could be detected at 280 nm in the flow through fraction. The so determined amount of protein was more than twice the amount of protein when compared to the single load of 7.5 mg/ml used in the flow through process according to the prior art using starting material 1. Using starting material 2 (filtrate of a 40% ammonium sulphate precipitate), the so determined amount was more than 4-fold the amount of protein when compared to the single load of 7.5 mg/ml used in the flow through process according to the prior art using starting material 1 (re-dissolved 60% ammonium sulphate precipitate).

Thereafter chromatography runs with respectively a single and a double load were respectively carried out in the flow through mode (i.e. as in the prior art) and in the binding and elution mode according to the present invention. A comparison of tris glycin gels made with samples of all four runs shows that the C1-INH peaks of the samples taken from the two runs according to the invention had a higher purity than the C1-INH peaks of samples taken from runs according to the prior art, and that irrespective of sample load, and that the least pure C1-INH peak was found in a double load run in the flow through mode according to the prior art. The results are shown in FIG. 3 discussed above.

Data of particular experiments are shown in the following table 2. Chromatograms corresponding to some of these experiments are shown in FIGS. 7 to 11 discussed above. Table 2 lists experiments carried out as in the prior art (“flow through”), according to the invention (“positive mode”) using aforementioned column A (with a column volume (CV) of 36 ml) or column B (with a column volume of 4.7 ml and respectively one of the following starting materials 1-4:
the same as in prior art EP 0 101 935, i.e. a re-dissolved 60% ammonium sulphate (AS) precipitate (=starting material 1);
the filtrate of an earlier 40% AS precipitate (=starting material 2),
lyophilised Berinert® product (=starting material 3),
the combined eluates of two HIC experiments using starting material 1 (=starting material 4).

The respective starting material is dissolved in the equilibration buffer. Elution takes place by means of a concentration and/or pH gradient at a specific amount of column volumes (CV), or via step elution, unless otherwise noted. Detection of C1-INH peaks is effected as described above.

TABLE 2

| | | | starting | equilibration | | | mS/cm | g/L AS | |
|---|---|---|---|---|---|---|---|---|---|
| experiment | chromatogram | column | material | buffer | elution | elution buffer | peak | peak | observation |
| 180418HW-1 | | A | 1 | AS = 106 g/L | gradient | AS = 106 g/L | | | flow- |
| | | | | pH = 6 | 10 CV | pH = 8.5 | | | through |
| 180418HW-2 | | A | 1 | AS = 106 g/L | gradient | AS = 106 g/L | | | flow- |
| | | | | pH = 8.5 | 10 CV | pH = 6 | | | through |
| 180419HW-1 | FIG. 7 | A | 1 | AS = 106 g/L | equilibration | AS = 106 g/L | | | flow- |
| | | | | pH = 7.2 | buffer | pH = 7.2 | | | through |
| 180419HW-2 | FIG. 8 | A | 1 | AS = 209 g/L | gradient | AS = 0 g/L | 151 | 160 | positive |
| | | | | pH = 7.2 | 10 CV | pH = 7.2 | | | mode |
| 180424HW-1 | FIG. 9 | A | 2 | AS = 181 g/L | gradient | AS = 0 g/L | | 160 | positive |
| | | | | pH = 7.2 | 10 CV | pH = 7.2 | | | mode |
| 180425HW-1 | | A | 2 | AS = 181 g/L | gradient | AS = 0 g/L | 156 | 167 | positive |
| | | | | pH = 7.2 | 10 CV but | pH = 7.2 | | | mode |
| | | | | | 50 cm/h | | | | |
| 180425HW-2 | | A | 2 | AS = 181 g/L | gradient | AS = 0 g/L | 159 | 171 | positive |
| | | | | pH = 7.2 | 20 CV | pH = 7.2 | | | mode |
| 180503HW | | A | 2 | AS = 181 g/L | gradient | AS = 112 g/L | 158 | 169 | positive |
| | | | | pH = 7.2 | 10 CV | pH = 7.2 | | | mode |
| 180508HW-2 | | B | 2 | AS = 181 g/L | gradient | AS = 0 g/L | 125 | 124 | positive |
| | | | | pH = 7.2 | 10 CV | pH = 7.2 | | | mode |
| 180516HW-2 | FIG. 10 | A | 2 | AS = 181 g/L | step | AS = 154 g/L | | | positive |
| | | | | pH = 7.2 | 5 CV | pH = 7.2 | | | mode |
| 180516HW-3 | | B | 4 | AS = 181 g/L | gradient | AS = 0 g/L | 129 | 129 | positive |
| | | | | pH = 7.2 | 10 CV | pH = 7.2 | | | mode |
| 180528HW | | B | 2 | AS = 181 g/L | gradient | AS = 0 g/L | 130 | 131 | positive |
| | | | | pH = 7.2 | 20 CV | pH = 7.2 | | | mode |
| 180619HW | FIG. 11 | A | 3 | AS = 292 g/L | gradient | AS = 0 g/L | 153 | 162 | positive |
| | | | (0.1 mg/ml) | pH = 7.2 | 10 CV | pH = 7.2 | | | mode |
| 180520HW | | A | 2 | AS = 181 g/L/ | loading | loading | | | loading |
| | | | (4-fold load) | pH = 7.2 | | | | | 30 mg |
| | | | | | | | | | protein/ml gel |
| 180626HW | | A | 1 | AS = 181 g/L/ | gradient | AS = 0 g/L | | | column |
| | | | (4-fold load) | pH = 7.2 | 20 CV | pH = 7.2 | | | over-loaded |
| 180627HW | | A | 1 | AS = 181 g/L | gradient | AS = 0 g/L | 161 | 174 | positive |
| | | | (2-fold load) | pH = 7.2 | 20 CV | pH = 7.2 | | | mode |
| 180628HW | | A | 1 | AS = 181 g/L | gradient | AS = 0 g/L | 156 | 167 | positive |
| | | | (single load) | pH = 7.2 | 20 CV | pH = 7.2 | | | mode |
| 180627HW | | A | 1 | AS = 181 g/L | gradient | AS = 0 g/L | | | column |
| | | | (3-fold load) | pH = 7.2 | 20 CV | pH = 7.2 | | | over-loaded |

As can be seen from Table 2, the ammonium sulphate (AS) concentration at which C1-INH elution peaks are observed is between about 160 and about 174 mg/ml when using column A, and between about 124 and about 131 mg/ml when using column B. As can further be seen, the loading capacity of column A when using starting material 1 is at least twice the single load, i.e. at least 2×7.5 mg or 15 mg protein/ml chromatography gel, and at least 4-fold the single load, i.e. at least 30 mg protein/ml chromatography gel, when using starting material 2.

Table 3 depicts a further experiment in which a large number of different gel types were compared. Under the conditions described in Table 3 C1-INH did bind to the matrix and was eluted with different gradients.

TABLE 3

| Manu- | | | | |
|---|---|---|---|---|
| facturer | Gel | Mode | Bindung | Elution |
| GE | Butyl HP | Binding and Elution | 181 g/L | Gradient from 181 zu 0 g/L |
| | | | ammonium sulphate | Ammonium sulphate |
| GE | Capto Butyl | Binding and Elution | 181 g/L | Gradient from 181 zu 0 g/L |
| | | | ammonium sulphate | Ammonium sulphate |
| GE | Phenyl HP | Binding and Elution | 181 g/L | Gradient from 200 zu 0 g/L |
| | | | ammonium sulphate | Ammonium sulphate |
| GE | Octyl FF | Binding and Elution | 181 g/L | Gradient from 181 zu 0 g/L |
| | | | ammonium sulphate | Ammonium sulphate |
| GE | Butyl-S FF | Binding and Elution | 181 g/L | Gradient from 181 zu 0 g/L |
| | | | ammonium sulphate | Ammonium sulphate |
| GE | Capto Phenyl | Binding and Elution | 181 g/L | Gradient from 200 zu 0 g/L |
| | ImpRes | | ammonium sulphate | Ammonium sulphate |
| GE | Octyl-S FF | Binding and Elution | 181 g/L | Gradient from 181 zu 0 g/L |
| | | | ammonium sulphate | Ammonium sulphate |
| GE | Capto Phenyl | Binding and Elution | 181 g/L | Gradient from 181 zu 0 g/L |
| | high sub | | ammonium sulphate | Ammonium sulphate |
| GE | Capto Butyl | Binding and Elution | 181 g/L | Gradient from 181 zu 0 g/L |
| | ImpRes | | ammonium sulphate | Ammonium sulphate |
| Tosoh | Butyl-600M | Binding and Elution | 181 g/L | Gradient from 181 zu 0 g/L |
| | | | ammonium sulphate | Ammonium sulphate |
| Tosoh | Phenyl-650M | Binding and Elution | 181 g/L | Gradient from 200 zu 0 g/L |
| | | | ammonium sulphate | Ammonium sulphate |
| Tosoh | Butyl-650M | Binding and Elution | 181 g/L | Gradient from 181 zu 0 g/L |
| | | | ammonium sulphate | Ammonium sulphate |
| Tosoh | PPG-600M | Binding and Elution | 181 g/L | Gradient from 181 zu 0 g/L |
| | | | ammonium sulphate | Ammonium sulphate |
| Tosoh | Phenyl-600M | Binding and Elution | 181 g/L | Gradient from 200 zu 0 g/L |
| | | | ammonium sulphate | Ammonium sulphate |
| Tosoh | TSKgel | Binding and Elution | 181 g/L | Gradient from 181 zu 0 g/L |
| | | | ammonium sulphate | Ammonium sulphate |
| GE | Capto Phenyl | Binding and Elution | 4M | Gradient from 4 zu 0 molar |
| | high sub | | Sodium chloride | sodium chloride |

In SDS gels (data not shown) the purity of the eluted C1-INH was analyzed and it was found that the 4 gel types depicted in Table 4 provided the best resolution of C1-INH from contaminating proteins. In a subsequent experiment using the binding and elution conditions depicted in Table 3 the yield of C1INH was compared between these 4 gel types and it was found that Phenyl-Hans-Peter® from GE Healthcare followed by Phenyl-650M® from Tosoh provided the best yield.

TABLE 4

| | | C1-INH |
|---|---|---|
| Manufacturer | Gel | Yield % |
| GE | Phenyl HP | 100% |
| GE | Capto Phenyl | 93% |
| | ImpRes | |
| Tosoh | Phenyl-650M | 97% |
| Tosoh | Phenyl-600M | 94% |

1. Process for purifying C1-INH using hydrophobic interaction chromatography, which comprises the steps of:
(i) loading a solution containing C1-INH dissolved therein onto a hydrophobic interaction chromatography column comprising a stationary phase under first conditions under which C1-INH binds to the stationary phase,
(ii) applying second conditions so as to elute C1-INH by means of a mobile phase.

2. Process according to claim 1, characterized in that
the first conditions are that the mobile phase comprises an anti-chaotropic salt, preferably sodium sulphate or ammonium sulphate, most preferably ammonium sulphate in a first concentration at which C1-INH binds to the stationary phase, and
the second conditions are that the mobile phase comprises the anti-chaotropic salt, preferably sodium sulphate or ammonium sulphate, most preferably ammonium sulphate in a second concentration at which C1-INH gets eluted.

3. Process according to claim 2, wherein transition from the first concentration to the second concentration is achieved by means of a concentration gradient, or by means of a step elution.

4. Process according to claim 2 or 3, wherein the stationary phase is chosen from one or more of the following matrix materials: agarose, cross-linked agarose (sold under various trade names, such as Sepharose®), hydrophilic polymers, e. g. polymethacrylate, substituted with hydrophobic ligands such as
linear alkyl, e.g. ethyl, butyl, octyl,
ramified alkyl, e.g. t-butyl,
aryl, e.g. phenyl, or
cycloalkyl, e.g. hexyl,
wherein the stationary phase is preferably a matrix material substituted with alkyl or aryl, preferably butyl or phenyl, and more preferably a cross-linked agarose substituted with butyl or phenyl, most preferably with phenyl.

5. Process according to claim 4, wherein the stationary phase is a phenyl substituted Sepharose® gel, such as Phenyl Sepharose® 6 Fast Flow (low sub) by GE Healthcare.

6. Process according to claim 5, wherein ammonium sulphate is used as chaotropic salt and the first concentration is above a concentration X in a range of about 1.1 M to about 1.4 M (e.g. above a concentration X in the range of about 155 to about 180 mg/ml ammonium sulphate), preferably in a range of about 1.2 M to about 1.3 M (e. g. above a concentration X in the range of about 160 to about 174 mg/ml ammonium sulphate), and wherein the second concentration is below concentration X.

7. Process according to claim 4, wherein the stationary phase is a butyl substituted Sepharose® gel, such as HiScreen™ Capto™ Butyl HP sold by GE Healthcare.

8. Process according to claim 7 wherein ammonium sulphate is used as chaotropic salt and the first concentration is above a concentration X in a range of about 0.9 M to about 1.0 M (e. g. a concentration X in the range of about 124 to about 131 mg/ml), and wherein the second concentration is below concentration X.

9. Process according to claim 4 wherein the stationary phase is Phenyl-HP® or Capto-Phenyl ImpRes® sold by GE Healthcare or Phenyl-650M® or Phenyl-600M® sold by Tosoh.

10. Process according to claim 9, wherein ammonium sulphate is used as chaotropic salt and the first concentration is above a concentration X in a range of about 1.1 M to about 1.4 M (e.g. above a concentration X in the range of about 155 to about 180 mg/ml ammonium sulphate), preferably in a range of about 1.2 M to about 1.3 M (e. g. above a concentration X in the range of about 160 to about 174 mg/ml ammonium sulphate), and wherein the second concentration is below concentration X.

11. Process according to any one of the preceding claims 2 to 10, wherein ammonium sulphate is used as chaotropic salt and the first concentration is between about 1.3 M to about 1.6 M, preferably between about 1.3 M to about 1.4 M, most preferably about 1.32 M (i.e. about 181 mg/ml).

12. Process according to any one of the preceding claims, wherein the C1-INH is recombinant C1-INH, transgenic C1-INH, or C1-INH derived from blood plasma, preferably human blood plasma.

13. Process according to any one of the preceding claims, wherein the C1-INH concentrate used as a starting material is obtained by a process involving a fractional precipitation with a precipitant.

14. Process according to claim 13, wherein the fractional precipitation does involve precipitation of C1-INH and wherein the C1-INH is taken up in a solution containing the precipitant at a concentration lower than necessary for a precipitation of C1-INH.

15. Process according to claim 14, wherein the fractional precipitation does not involve precipitation of C1-INH and wherein the C1-INH is contained within a supernatant containing the precipitant at a concentration lower than necessary for a precipitation of C1-INH.

16. Process according to any one of the preceding claims, wherein the process is carried out at a pH in the range of 6 to 9, preferably 6.8 to 8.5, more preferably 7 to 7.5, and even more preferably at a pH of about 7.2.
